(19) United States
(12) Patent Application Publication
(10) Pub. No.: US 2012/0142618 A1
(43) Pub. Date: Jun. 7, 2012

(54) KETOGENIC SACCHARIDES

(75) Inventor: Richard A. Gross, Brooklyn, NY (US)

(73) Assignee: BTG International Limited, London (GB)

(21) Appl. No.: 13/333,626

(22) Filed: Dec. 21, 2011

Related U.S. Application Data
(63) Continuation of application No. 11/658,154, filed on Feb. 13, 2007, now abandoned.

(51) Int. Cl. A61K 31/7024; A61P 25/28; A61P 3/10; A61P 3/04; A61P 25/00; A61P 9/10; A61P 9/04; C07H 13/04; A61P 25/08

(52) U.S. Cl. 514/23; 536/119

(57) ABSTRACT

A novel ketogenic compound is provided having general formula (R(OCH(CH₃)CH₂C(O))ₙO)ₘ-A wherein n is a integer between I and 10, m is an integer of 1 to 200,000, A is a monosaccharide, polysaccharide or oligosaccharide residue and R is selected from the group consisting of H, C₁-C₆ alkyl and acetoacetyl-

The present invention relates to novel compounds which have utility as nutraceuticals and medicaments for producing ketosis in humans and animals for nutraceutical or therapeutic purposes.

It is known that ketone bodies, particularly (R)-3-hydroxybutyrate (D-β-hydroxybutyrate) and acetoacetate have both nutritional and therapeutic application in man and many animals. U.S. Pat. No. 6,136,862 and U.S. Pat. No. 6,232,345 (incorporated herein by reference) relate to the use of D-β-hydroxybutyrate, oligomers, esters and salts thereof, inter alia, in the treatment of cerebral edema and cerebral infarction. U.S. Pat. No. 6,207,856 and PCT/US99/21015 also refer to β-hydroxybutyrate and its oligomers, esters and salts thereof in protecting against other forms of neurodegeneration inter alia, through their proposed ability to activate the TCA cycle and, through favourable redox reactions in cells and antioxidant activity, scavenge free radicals. β-hydroxybutyrate has also been demonstrated to have cardioprotectant effect and can increase cardiac efficiency (Sato et al FASEB J 9: 651-658, 1995).

U.S. Pat. No. 6,207,856, U.S. Pat. No. 6,136,862, U.S. Pat. No. 6,207,856 and PCT/US99/21015, incorporated herein by reference, teach that preferred ketogenic precursors for producing such ketosis are monohydric-, dihydric and trihydric alcoholic esters of (R)-3-hydroxybutyrate, but particularly a (R)-3-hydroxybutyryl ester of (R)-1,3-butandiol, more preferably the diester formed from two molecules of (R)-3-hydroxybutyrate and one molecule of (R)-1,3-butandiol.

However, it is also known that production of oligomers of (R)-3-hydroxybutyrate in pure form is problematic. PCT/US99/21015 exemplifies a ketogenic oligomer with a mixture of (R)-3-hydroxybutyrate timer, tetramer and pentamer and exemplifies esters thereof with acetoacetyl trimer, tetramer and pentamer of (R)-3-hydroxybutyrate. Similarly, Hiraide et al al (1999) J. Parenteral and Enteral Nutrition Vol 23. No 6 discloses use of a mixture of dimer and trimer of (R)-3-hydroxybutyrate for studies in ability of plasma to degrade these to the monomer.

Copending provisional patent applications of Richard Gross (U.S. provisional filings 60/5,883,156 and U.S. 60/588,990) claim compounds comprising fixed length oligomers of (R)-3-hydroxybutyrate esterifed to monohydric and dihydric alcohols, methods for synthesising these in pure form and methods of treatment using these. These compounds are either water soluble syrups or water insoluble waxy solids.

Henderson et al WO2004077938, postulate use of saccharides which have been substituted on all of their free hydroxyl groups by fatty acyl, acetoacetyl or hydroxybutryl groups as compounds for producing ketosis in subjects in need of ketogenic therapy. The formulae taught in that document are fully substituted, no synthetic routes are discussed and no biological date provided.

The present inventors have now determined that in order to produce useful ketosis in a subject it is in fact necessary to use saccharides that are not fully substituted by ketogenic precursor moieties. It is believed that it is important that some significant level of hydroxylation remain on the saccharide in order for efficient metabolism of the compounds to proceed and useful ketosis occur.

The present invention provides a ketogenic saccharide material which is suitable for use in animals and man for therapeutic purposes. Preferred compounds of the present invention are soluble in water and other aqueous liquids and therefor have application in beverages and liquid, semi-solid or gelled orally administerable medicaments. Preferred compounds are of single component constituent.

In a first aspect of the present invention there is provided a compound of general formula

(R(OCH(CH₃)CH₂C(O))ₙO)ₘ-A

wherein n is a integer between 1 and 10, m is an integer of 1 to 200,000, A is a monsaccharide, oligosaccharide or polysaccharide residue and R is selected from the group consisting of H, C₁-C₆ alkyl and acetoacetyl-,

wherein m is such that the number of free hydroxyl groups on the compound is at least an average of 0.3 free hydroxyls per saccharide moiety in residue A.

Thus the group R(OCH(CH₃)CH₂C(O))ₙO— is esterified to the moiety A by substituting for the H on a number ‘m’ of saccharide hydroxy groups. N is preferably 1 to 3.

Preferably m is an integer of 1 to 20,000 more preferably 1 to 200, still more preferably 1 to 100, eg. 3 to 100. Clearly the precise number ‘m’ will depend upon whether the compound is a monosaccharide, where m cannot be more than 4 or 5 for hexoses and heptoses; an oligosaccharide, where in cannot be more than 3 or 4 for hexoses and heptoses. Where the saccharide is a polysaccharide m is proportionately able to be a multiple of the number of monomers in the polymer.

Preferably there is at least one free hydroxyl group in the compound for each saccharide ring in the compound. It will be realised that this may be an average number of hydroxyl groups, wherein some rings will have no free hydroxyls on each saccharide ring of the compound whilst others have more than one. In a preferred group of compounds at least one hydroxyl group on each ring remains unsubstituted.

In one preferred aspect of the present invention A is a monosaccharide, oligosaccharide or polysaccharide and m is equal the number of repeat sugar monomer moieties in the saccharide multiplied by a substitution factor (aka degree of substitution) of between 0 5 and 4: the substitution factor being an indication of the average number of the free hydroxyl groups situated on each saccharide moiety of monosaccharide, oligosaccharide or polysaccharide, ie. that have been substituted; more preferably being a number of between 0.6 and 4 for every saccharide moiety in the molecule, more typically between 1 and 3, eg. 1 and 2 for every such moiety.

Preferred monosaccharides are tetroses, pentoses, hexoses, heptoses; preferred oligosaccharides are disaccharides and higher oligomers of these the monosaccharides. Preferred polysaccharides are those used in foodstuffs, particularly preferred being glucose based saccharides, eg pullulans. Pullulan is a linear homopolysaccharide of glucose that is an α-(1-6)-linked polymer of maltotriose subunits. It has adhesive properties and is suitable for forming a variety of forms and derivatises easily such that its solubility can be controlled.

In a second aspect of the present invention there is provided a nutraceutrial or pharmaceutical composition comprising a compound of the first aspect together with a foodstuff component or a pharmaceutically acceptable carrier, diluent or excipient. Suitable foodstuff components may, but are not limited to, edible oils, emulsions, gels or solids and drinkable liquids, including suspensions and solutions.

In a third aspect of the present invention there is provided the use of a compound of the first aspect of the present invention for the manufacture of a medicament for producing a physiologically acceptable ketosis. Such medicament will be suitable for treating a number of debilitating conditions, including trauma, haemorrhagic shock, neurodegeneration, diabetes, and epilepsy, stroke, head trauma, myocardial infarction, congestive heart failure, pulmonary failure, kidney failure and obesity.

In a fourth aspect of the present invention there is provided a method for the manufacture of a compound of formula

(R(OCH(CH₃)CH₂C(O))ₙO)ₘ-A

wherein n is a integer between 1 and 30, m is an integer of 1 to 200,000, A is a monsaccharide, polysaccharide or oligosaccharide residue and R is selected from the group consisting of H, C₁-C₆ alkyl and acetoacetyl-

wherein m is such that the number of free hydroxyl groups on the compound is at least an average of 0.3 free hydroxyls per saccharide moiety in residue A.

comprising
reacting (R)-3-hydroxybutyrate or an oligomer thereof containing between 2 and 10 (R)-3-hydroxybutyrate moieties with a monosaccharide, oligosaccharide or polysaccharide in the presence of an acid and in an organic solvent.

Preferably the solvent provides the acid; more preferably then solvent is an organic acid, more particularly being toluene sulphonic acid, eg. Para-toluene sulphonic acid.

The reaction mixture may advantageously also include dimethylsulphoxide.

In a fifth aspect of the present invention there is provided a method for the manufacture of a compound of formula

R(OCH(CH₃)CH₂C(O))ₙO)ₘ-A

wherein n is a integer between 1 and 10, m is an integer of 1 to 200,000, A is a monsaccharide, polysaccharide or oligosaccharide residue and R is selected from the group consisting of H, C₁-C₆ alkyl and acetoacetyl-

wherein m is such that the number of free hydroxyl groups on the compound is at least an average of 0.3 free hydroxyls per saccharide moiety in residue A.

comprising
reacting (R)-3-hydroxybutyrate or an oligomer thereof containing between 2 and 10 (R)-3-hydroxybutyrate moieties with a monosaccharide, oligosaccharide or polysaccharide in the presence of dimethylsulphoxide (DMSO) in an organic solvent.

Preferably the solvent is DMSO.

The compounds where n is more than 1 may be made alternately by reacting a monosaccharide, oligosaccharide or polysaccharide having been substituted with H(OCH(CH₃)CH₂C(O))ₘO—, wherein n is 1, with a cyclic oligomer of (R)-hydroxybutyrate in the presence of a lipase, a reaction disclosed in my copending provisional applications related to esterification of mono-ols and diols. Such reaction is conveniently carried out in THF with Novozym 435 (a CAL B enzyme). Where a trimer of (R)-3-hydroxybutyrate is to be added to the HOCH(CH₃)CH₂C(O)O-substituted saccharide, the triolide of (R)-3-hydroxybutyrate is employed.

Compounds where R is C₁-C₆ alkyl and acatoacetyl can be made from the corresponding compound where R is H by simple esterification with the acetoacetate or use of an alkylating agent.

Regarding starting materials for producing the compounds of the present invention, various cyclic esters of (R)-3-hydroxybutyrate are known in the art and are readily produced by known methods: see for example see for example Seebach et al. Helvetia Chimica Acta Vol 71 (1988) pages 155-167, and Seebach et al. Helvetia Chimica Acta, Vol 77 (1994) pages 2007 to 2033.

The present invention will now be described further by reference to the following non-limiting Examples, Schemes and Figures. Further embodiments falling within the scope of the claim will occur to those skilled in the art in the light of these.

### FIGURES

FIG. 1: General scheme showing the synthesis of KTX 0310 by the esterification of glucose with (R)-3-hydroxybutyric acid in the presence of CAL-B.

FIG. 2: General scheme showing the synthesis of KTX 0311 by the esterification of fructose with (R)-3-hydroxybutyric acid in the presence of CAL-B.

FIG. 3: General scheme showing the synthesis of KTX 0312 by the esterification of arabinose with (R)-3-hydroxybutyric acid in the presence of CAL-B.

FIG. 4: General scheme showing the synthesis of KTX 0313 by the esterification of sorbitol with (R)-3-hydroxybutyric acid in the presence of CAL-B.

FIG. 5: General scheme showing the synthesis of KTX 0301 and poly(3-hydroxybutyrate) oligomers by the esterification of pullulan with (R)-3-hydroxybutyric acid in the presence of para-toluene sulphonic acid.

FIG. 6: General scheme showing the synthesis of KTX 0321 by the esterification of pullulan with (R)-3-hydroxybutyric acid in the presence of para-toluene sulphonic acid and dimethylsulphoxide

FIG. 7: General scheme showing the synthesis of KTX 0322 by the esterification of soluble starch with (R)-3-hydroxybutyric acid in the presence of para-toluene sulphonic acid.

FIG. 8: Effect of oral administration KTX 0310 (glucose (R)-3-hydroxybutyrate ester) as determined by increases of β-hydroxybutyrate concentrations in rat plasma.

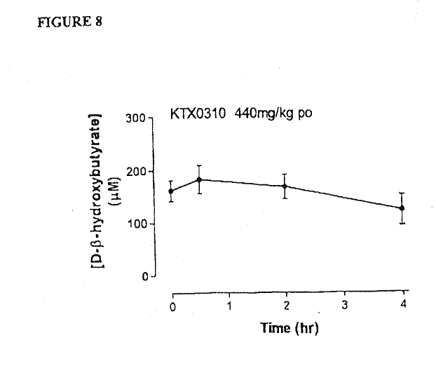

FIG. 9: Effect of oral administration KTX 0311 (fructose (R)-3-hydroxybutyrate ester) as determined by increases of β-hydroxybutyrate concentrations in rat plasma.

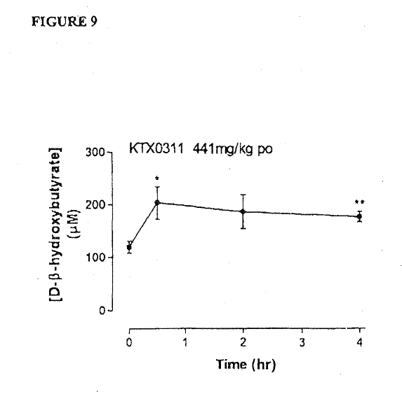

FIG. 10: Effect of oral administration KTX 0312 (arabinose (R)-3-hydroxybutyrate ester) as determined by increases of β-hydroxybutyrate concentrations in rat plasma.

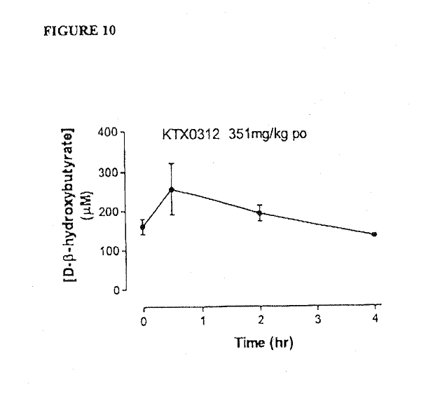

FIG. 11: Effect of oral administration KTX 0313 (the sorbitol tn-ester) as determined by increases of β-hydroxybutyrate concentrations in rat plasma.

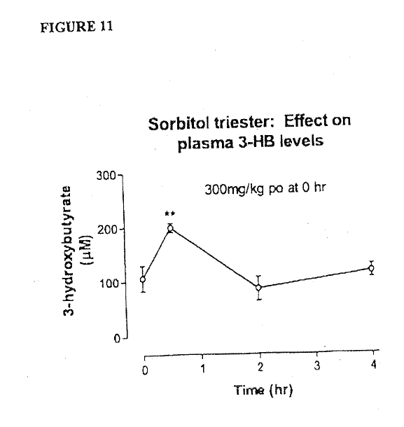

FIG. 12: Effect of oral administration KTX 0301 (a pullulan (R)-3-hydroxybutyrate ester+PHB oligomers) as determined by increases of β-hydroxybutyrate concentrations in rat plasma.

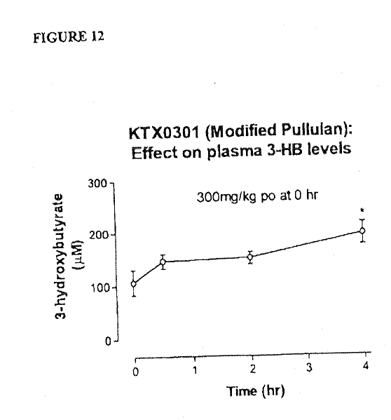

FIG. 13: Effect of oral administration KTX 0321 (a purified pullulan (R)-3-hydroxybutyrate ester) as determined by increases of β-hydroxybutyrate concentrations in rat plasma.

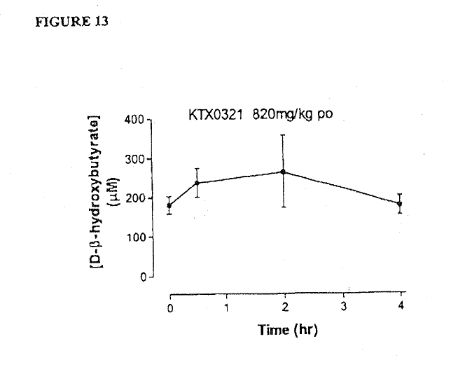

FIG. 14: Effect of oral administration KTX 0322 (a purified soluble starch (R)-3-hydroxybutyrate ester) as determined by increases of β-hydroxybutyrate concentrations in rat plasma.

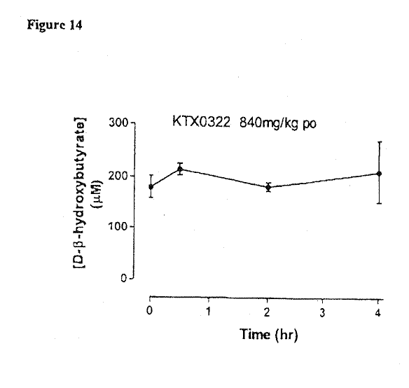

### EXAMPLES

Procedure for the Synthesis of Methyl [R]-3-Hydroxybutyrate

To a 1 L, one-neck round bottom flask equipped with condenser and magnetic stirring was charged 62.5 g poly([R]-3-hydroxybutyrate) (PHB) Julich, Germany, and 350 ml 1,2-dichloroethane. A solution of acidic methanol was prepared by the careful addition of 12.5 mL con. H₂SO₄ to 250 mL methanol and this was added to the reaction slurry. Stirring was maintained with heating at 80° C. (reflux) for 120 hrs. The slurry was cooled to room temperature, extracted with 200 ml ½ saturated NaCl and 50 mL saturated NaCl. The organic material was re-extracted with 100 ml NaHCO₃ to a pH 6.0, followed by 2×50 ml saturated NaCl. The organic material was dried over MgSO₄, filtered, and the solvent was removed by rotary evaporation. The organic material was fractionally distilled at 0.3 mmHg, 45° C. to give 46 g (73% yield based on the initial polymer charge) of a clear colorless liquid. NMR was used to characterize the product.

Procedure for the Synthesis of [R]-3-Hydroxybutyric acid

Into a 50 ml flask was added 5N KOH (10 ml) which was cooled to 0° C. Methyl [R]-Hydroxybutyrate (5 g) was then added with stirring over 1.5-h time period, and the temperature was maintained at 0° C. for 24 h. The reaction was terminated by the slow addition of 6N HCl (8.3 ml) with stirring at 5° C. The resultant aqueous solution was then saturated with solid NaCl and extracted 20 times with 20 mL portions of diethyl ether. The organic extract was dried over anhydrous MgSO₄ and the ether removed by rotary evaporation. The product was a white crystalline solid (3.8 g, yield 87%). NMR and IR were used to characterize the product.

### Example 1

### The Synthesis of KTX 0310 by the Esterification of Glucose with (R)-3-hydroxybutyric acid in the Presence of CAL-B

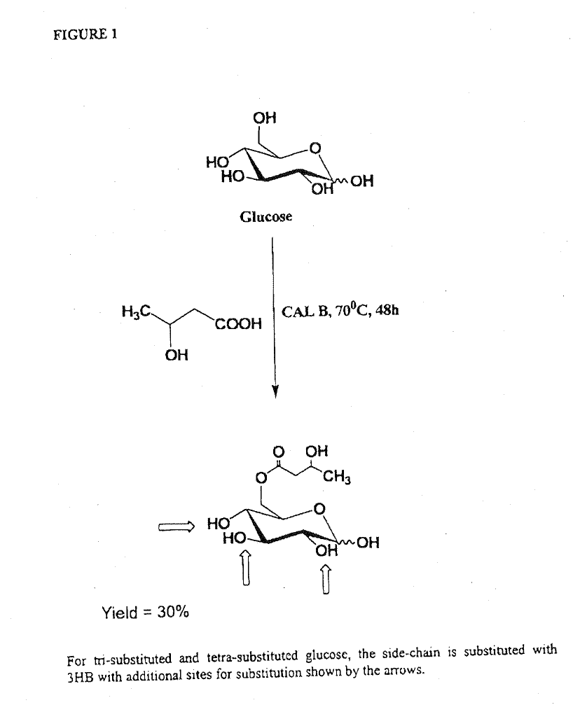

To a round-bottomed flask, 1 g glucose and 4.6 g 3-hydroxybutyric acid were added. The mixture was heated at 80° C. to obtain a homogenous solution. The temperature was lowered to 70° C. and 1.1 g (20% w/v of the total mixture) CAL B was added. The mixture was stirred at 70° C. for 48 hrs to yield glucose 3-hydroxybutyrate tri- and tetra-esters as shown in FIG. 1.

The material was separated by column chromatography based on its polarity. The column was packed in pure chloroform and the polarity was increased using methanol. The desired product was eluted using chloroform:methanol:water (9:2:0.3).

The product was a water-soluble syrup and was obtained at a yield of 0.3 g (30%). A mixture of tri- and tetra-substituted products was formed (substitution factor between 3 and 4 with 1 to 2 free hydroxyls left per monosaccharide ring). The structure of the compound was verified by LC/MS.

### Example 2

### The Synthesis of KTX 0311 by the Esterification of Fructose with (R)-3-Hydroxybutyric acid in the Presence of CAL-B

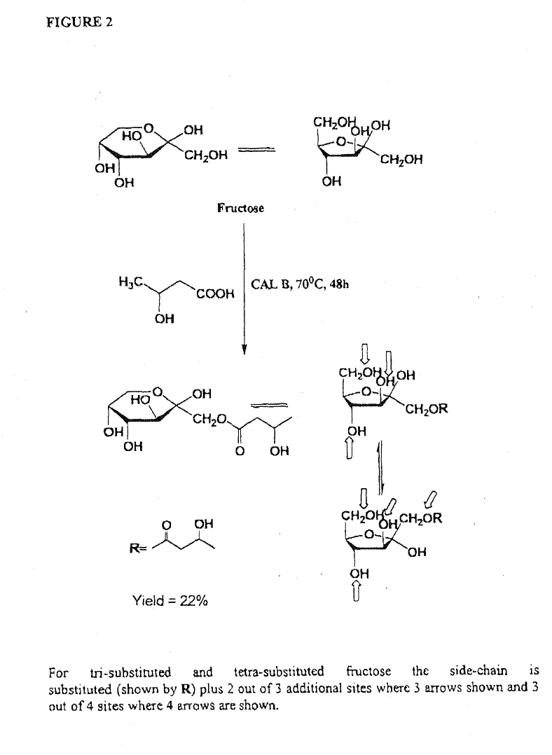

To a round-bottomed flask, 5 g fructose and 23 g 3-hydroxybutyric acid were added. The mixture was heated at 80° C. to obtain a homogenous solution. The temperature was lowered to 70° C. and 5.6 g (20% w/v of the total mixture) CAL B was added. The mixture was stirred at 70° C. for 48 hrs to yield the fructose 3-hydroxybutyrate tri- and tetra-esters as shown in FIG. 2.

The material was separated by column chromatography based on its polarity. The column was packed in pure chloroform and the polarity was increased using methanol. The desired product was eluted using chloroform:methanol:water (9:2:0.3).

The product was a water-soluble syrup and was obtained at a yield of 1.1 g (22%). A mixture of tri- and tetra-substituted products was formed (substitution factor between 3 and 4-1 to 2 free hydroxyls let on the monosaccharide ring). The structure of the compound was verified by LC/MS.

### Example 3

### The Synthesis of KTX 0312 by the Esterification of Arabinose with (R)-3-hydroxybutyric acid in the Presence of CAL-B

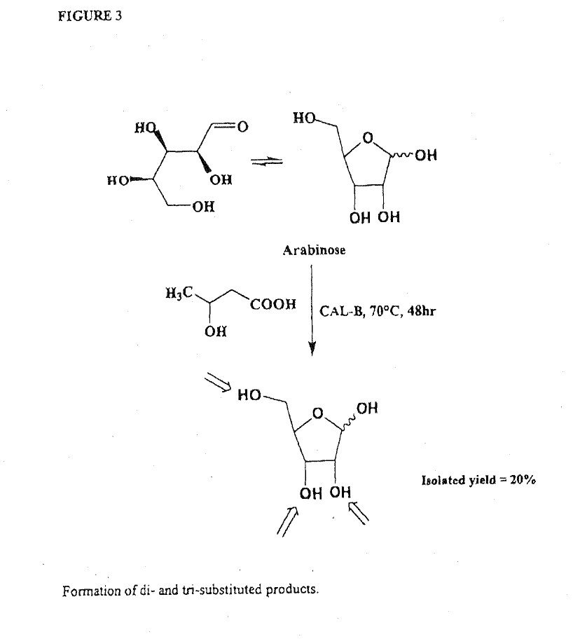

To a round-bottomed flask, 1 g arabinose and 5.5 g 3-hydroxybutyric acid were added. The mixture was heated at 80° C. to obtain a homogenous solution. The temperature was lowered to 70° C. and 1.3 g (20% w/v of the total mixture) CAL B was added. The mixture was stirred at 70° C. for 48 hrs to yield the arabinose 3-hydroxybutyrate di- and tri-esters as shown in FIG. 3.

The material was separated by column chromatography based on its polarity. The column was packed in pure chloroform and the polarity was increased using methanol. The desired product was eluted using chloroform:methanol:water (9:2:0.3).

The product was a water-soluble syrup and was obtained at a yield of 0.2 g (20%). A mixture of di- and tri-substituted products was formed (substitution factor 2 to 3 leaving 1 to 2 free hydroxyls per monosaccharide moiety. The structure of the compound was verified by LC/MS and by ¹H NMR (300 MHz, CDCl₃) and ¹³C NMR (75.5 MHz, CDCl₃) spectroscopy.

### Example 4

### The Synthesis of KTX 0313 by the Esterification of Sorbitol with (R)-3-hydroxybutyric acid in the presence of CAL-B

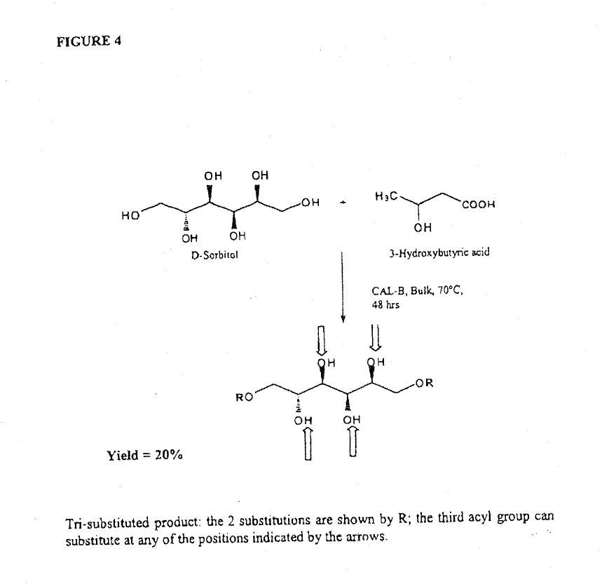

To a round-bottomed flask, 5 g sorbitol and 8.6 g 3-hydroxybutyric acid were added. The mixture was heated at 80° C. to obtain a homogenous solution. The temperature was lowered to 70° C. and 2.7 g (20% w/v of the total mixture) CAL B was added. The mixture was stirred at 70° C. for 48 hrs to yield the sorbitol 3-hydroxybutyrate tri-ester as shown in FIG. 4.

The material was separated by column chromatography based on its polarity. The column was packed in pure chloroform and the polarity was increased using methanol. The desired product was eluted using chloroform:methanol:water (9:2:0.3).

The product was a water-soluble syrup and was obtained at a yield of 1 g (20%). The product had a degree of substitution of 3, (leaving 3 free hydroxyls per monosaccharide moiety). The structure of the compound was verified by MALDI mass spectrometry and ¹H NMR (300 MHz, CDCl₃).

### Example 5

### The Synthesis of KTX 0301 and oligo(3-hydroxybutyrate) Oligomers by the Esterification of Pullulan with (R)-3-hydroxybutyric acid in the Presence of para-toluene sulphonic acid

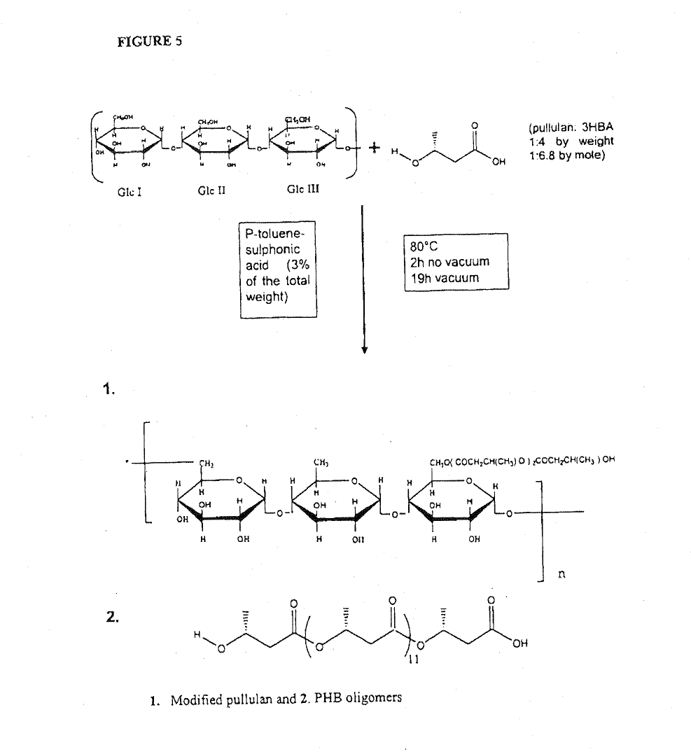

To a 100 ml round-bottomed flask were added with constant stirring, 5.3 g pullulan, 21.2 g (R)-3-hydroxybutyric acid and 79.5 mg p-toluene sulphonic acid. The flask was capped with a rubber septum and vacuum and dry nitrogen were applied alternately to the flask via a 3-way connector to remove any moisture and to fill the flask with dry nitrogen. The flask contents were heated to a constant 80° C. in an oil bath with continuous stirring. Pullulan was dispersed in the melt of (R)-3-hydroxybutyric acid. After 2 hrs, the reaction mixture was stirred under vacuum for 19 hrs. The reaction mixture was then ground to a fine powder and stirred in acetone overnight. The mixture was filtered, washed with acetone and then dried under reduced pressure at room temperature. The scheme for the synthesis of KTX 0301 and its chemical structure are shown in FIG. 5.

Yield: 8.5 g of water-insoluble grey solid. The structure of the acylated pullulan was determined by ¹H NMR (300 MHz, CDCl₃) and ¹³C NMR (75.5 MHz, CDCl₃) spectroscopy. The degree of esterification of pullulan by (R)-3-hydroxybutyrate was 0.33, leaving relatively high amounts of free hydroxyl groups on the KTX0301 compound. The oligomers of (R)-3-hydroxybutyrate contained in the mixture had an average degree polymerisation of 13. The mixture contained by weight: 33% 3-hydroxybutyric acid, 12% esterified pullulan and 21% PHB oligomers.

### Example 6

### The Synthesis of KTX 0321 by the Esterification of Pullulan with (R)-3-hydroxybutyric acid in the Presence of para-toluene sulphonic acid and dimethylsulphoxide

To a 100 ml round-bottomed flask were added with constant stirring, 7.75 g pullulan and 19.4 ml anhydrous dimethylsulphoxide (DMSO). The flask was capped with a rubber septum and vacuum and dry nitrogen were applied alternately to the flask via a 3-way connector to remove any moisture and to fill the flask with dry nitrogen. The flask contents were heated to a constant 80° C. in an oil bath with continuous stirring.

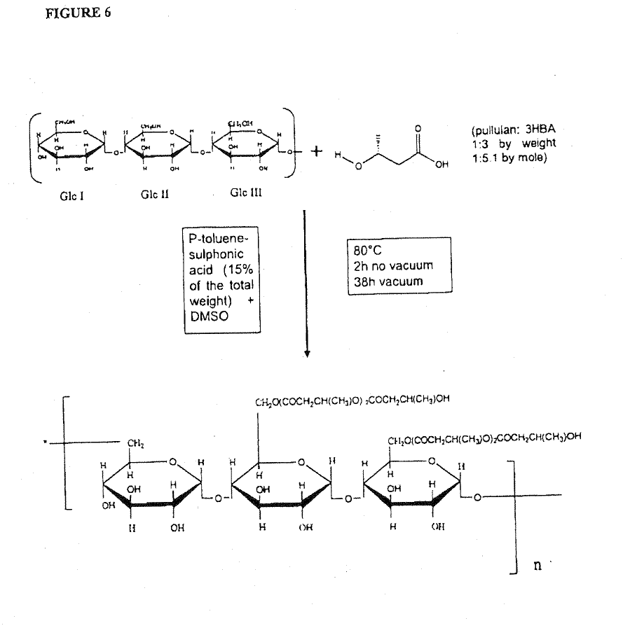

The flask was cooled to room temperature and 24.0 g (R)-3-hydroxybutyric acid and 1.16 g p-toluene sulphonic acid were added to the mixture. The flask was capped with a rubber septum and vacuum and dry nitrogen were applied alternately to the flask via a 3-way connector to remove any moisture and to fill the flask with dry nitrogen. The mask contents were heated to a constant 80° C. n an oil bath with continuous stirring. After the solution had had become clear, the reaction mixture was kept under vacuum for 38 hrs. The reaction mixture was added to a large amount of acetone with stirring and the precipitate was separated by centrifugation. More acetone was added to the precipitate and the centrifugation step was repeated several times. The product was then dried wider reduced pressure at room temperature for 3 days. The scheme for the synthesis of KTX 0321 and its chemical structure are shown in FIG. 6.

Yield: 5.1 g of water-insoluble white solid. The structure of the acylated pullulan was determined by ¹H NMR (300 MHz, CDCl₃) spectroscopy. The degree of substitution of pullulan by (R)-3-hydroxybutyrate was 0.64. Elemental analysis: C=43.33%, H=6.4% by weight.

### Example 7

### The Synthesis of KTX 0322 by the Esterification of Soluble Starch with (R)-3-hydroxybutyric acid in the Presence of para-toluene sulphonic acid

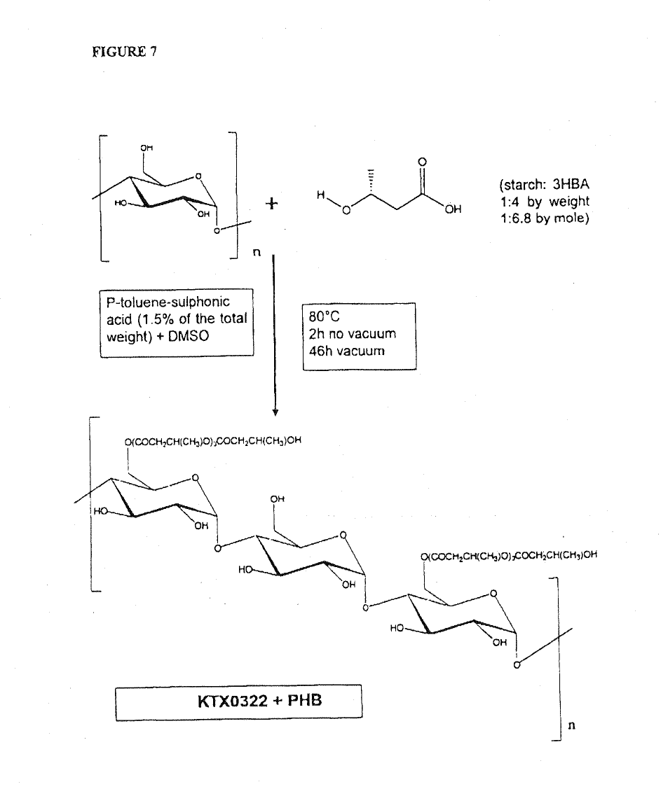

To a 100 ml round-bottomed flask were added with constant stirring 7.75 g soluble starch and 35 ml anhydrous dimethylsulphoxide (DMSO). The flask was capped with a rubber septum and vacuum and dry nitrogen were applied alternately to the flask via a 3-way connector to remove any moisture and to fill the flask with dry nitrogen. The flask contents were heated to a constant 80° C. in an oil bath with continuous stirring. The flask was cooled to room temperature and 24.0 g (R)-3-hydroxybutyric acid and 1.16 g p-toluene sulphonic acid were added to the mixture. The flask was capped with a rubber septum and vacuum and dry nitrogen were applied alternately to the flask via a 3-way connector to remove any moisture and to fill the flask with dry nitrogen. The flask contents were heated to a constant 80° C. in an oil bath under vacuum for 46 hrs. The reaction mixture was added to a large amount of acetone with stirring and the precipitate was separated by centrifugation. More acetone was added to the precipitate and the centrifugation step was repeated several times. The product was then dried under reduced pressure at room temperature for 3 days. The scheme for the synthesis of KTX 0322 and its chemical structure are shown in FIG. 7.

Yield: 3.9 g of water-insoluble white solid. The structure of the acylated soluble starch was determined by ¹H NMR (300 MHz, CDCl₃) spectroscopy. The degree of substitution of pullulan by (R)-3-hydroxybutyrate was 0.60. Elemental analysis: C=43.94%, H=6.49% by weight.

### Example 8

### Modification of Starch with [R]-3-Hydroxybutyric Acid

Procedure for Modification of Starch Nanospheres with [R]-3-Hydroxybutyric Acid

250 mg of starch nanosphere (from Ecosynthetix), 1.0 g of [R]-3-hydroxybutyric acid and 37.5 mg p-toluene-sulfonic acid were added to a 50 mL flask with stirring bar. The flask was capped by a rubber septum. Vacuum and dry nitrogen were applied to the flask alternately via three way connector to remove any moisture and fill the flask with dry nitrogen. The flask was placed into a constant temperature (80° C.) oil bath with stirring. After 2 h, the reaction mixture was subjected to reduced pressure for 19 h. The reaction product(s) were ground to a fine powder and stirred in acetone for 2 h. The mixture was then filtered. The white solid was washed with acetone and then dried under reduced pressure at room temperature to give a product yield of 408 mg. NMR, IR was used to characterize the product. The degree of substitution was measured as 1.2.

### Example 9

### Modification of Soluble Starch with [R]-3-Hydroxybutyric Acid

Soluble Starch, an was sourced as A.C.S. reagent, from Sigma-Aldrich. The method of esterification used was that of Example 1. NMR was used to characterize the product. The degree of substitution attained was 0.7.

### Example 10

### Modification of Pullulan with [R]-3-Hydroxybutyric Acid

Pullulan was sourced from Pfanstiehl Laboratories, Inc. The method of esterification used was that of Example 1. NMR was used to characterize the product. The degree of substitution was measured as 1.1

### Example 11

### Modification of Pectin and with [R]-3-Hydroxybutyric Acid

Pectin, from citrus fruits, was sourced ordered from Sigma. The method of Example 8 was used to modify these polysaccharides. The product was water soluble indicative of a low degree of substitution.

### Example 12

### Modification of Locust Bean Gum

Locust bean gum was treated as described in Example 1. The product was water soluble indicating a low degree of substitution.

### Example 13

### Ketogenesis in Rats In Vivo

Male Sprague-Dawley rats (weight range 200-250 g Charles River, Margate, Kent) were group housed in polypropylene cages at a temperature of 21±4° C. and 55±20% humidity and on a standard light/dark cycle. Animals had free access to a standard pelleted rat diet and tap water at all times. Animals were accustomed to these conditions for at least one week before experimentation.

Compounds were administered by oral gavage (po). Control animals received the appropriate vehicle via the same route. The experiment was performed over 2 days (ie 2 compounds were tested per day). Blood samples were taken by cardiac puncture after the animals were killed by a British Home Office Schedule 1 method. The terminal blood sample was collected into suitable plasma preparation tubes (EDTA-coated tubes). Plasma samples were initially frozen on dry ice and transferred to a −75° C. freezer until required for subsequent analysis (spectrophotometric analysis of (R)-3-hydroxybutyrate).

Protocol for KTX 0301, KTX 0313 Only

| | Number of | Time of blood | |
|---|---|---|---|
| Group | animals | sampling (min) | Treatment |
| A | 4 | 0 | Vehicle baseline |
| B | 4 | 30 | KTX 0301 (300 mg/kg po) |
| | | | or |
| | | | KTX 0313 (300 mg/kg po) |
| C | 4 | 120 | KTX 0301 (300 mg/kg po) |
| | | | or |
| | | | KTX 0313 (300 mg/kg po) |
| D | 4 | 240 | KTX 0301 (300 mg/kg po) |
| | | | or |
| | | | KTX 0313 (300 mg/kg po) |

Protocol for KTX 0311 Only

| | Number of | Time of blood | |
|---|---|---|---|
| Group | animals | sampling (mm) | Treatment |
| A | 4 | 0 | Vehicle baseline |
| B | 4 | 30 | KTX 0311 (441 mg/kg po) |
| C | 4 | 120 | KTX 0311 (441 mg/kg po) |
| D | 4 | 240 | KTX 0311 (441 mg/kg po) |

Protocol for KTX 0321, KTX 0310, KTX 0312 and KTX 0322

| | Number | Time of blood | |
|---|---|---|---|
| Group | of animals | sampling (mm) | Treatment |
| A | 8 | 30, 120, 240 | Vehicle (n = 2-3 for each time- |
| | | | point) |
| B | 4 | 30 | KTX 0321 (820 mg/kg po) |
| | | | or |
| | | | KTX 0310 (440 mg/kg po) |
| | | | or |
| | | | KTX 0312 (351 mg/kg po) |
| | | | or |
| | | | KTX 0322 (840 mg/kg po) |
| C | 4 | 120 | KTX 0321 (820 mg/kg po) |
| | | | or |
| | | | KTX 0310 (440 mg/kg po) |
| | | | or |
| | | | KTX 0312 (351 mg/kg po) |
| | | | or |
| | | | KTX 0322 (840 mg/kg po) |
| D | 4 | 240 | KTX 0321 (820 mg/kg po) |
| | | | or |
| | | | KTX 0310 (440 mg/kg po) |
| | | | or |
| | | | KTX 0312 (351 mg/kg po) |
| | | | or |
| | | | KTX 0322 (840 mg/kg po) |

Sodium DL-β-hydroxybutyrate (H-6501 Lot 111K2618) was obtained from Sigma. A stock solution of β-hydroxybutyrate (40 mM DL racemate equivalent to 20 mM D-isomer) was prepared in 0.9% saline solution, kept at 4° C. and used to generate appropriate dilutions for an assay standard curve. Such solutions have been shown to be stable for at least 2 months.

Commercial clinical assay kits for the determination of D-β-hydroxybutyrate were obtained from Randox Laboratories (Antrim, UK), Kits were obtained in two pack sizes (Ranbut RB1007: 10×10 ml RBI 008: 10×50 ml) but were otherwise identical. Each kit contained a standard solution of 1 mM D-β-hydroxybutyrate that was assayed every time to confirm the assay was performing correctly. The kit relies on measuring the appearance of NADH via the activity of β-hydroxybutyrate dehydrogenase measured as an increase of OD340 nm. An alkaline pH is necessary to drive the reaction equilibrium towards the production of NADH and acetoacetate;

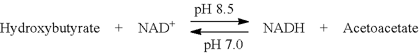

The protocol supplied with the Ranbut kits was for a discrete (cuvette-based) spectrophotometric assay, so the protocol was modified for suitability with a 96-well microplate format using blank, flat-bottomed microplates (Greiner PS 655101 Lot 98 35 01). Assays were performed in triplicate using a sample volume of 10 μl to each well for the standards and usually 20 μl for plasma samples (though this was varied for some experiments). Standard dilutions and samples were pipetted a single plate at a time and preincubated at 37° C. for 15 minutes in the sample compartment of a Molecular Devices VERSAₘₐₓ tunable microplate reader: The appropriate volume of assay reagent was reconstituted, according to instructions, using distilled water and preincubated at 37° C. for 15 minutes using a static water bath. The assay plate was ejected and the reaction started by adding rapidly 250 μl of reagent to each well (avoiding air bubbles). The plate was reloaded, mixed and then the change in OD340 nm followed in kinetic mode with a reading at every 15 seconds for a total of 2 minutes. The reaction rate was then determined from the OD increase over a suitable 1 minute period, after allowing a necessary period for the reaction rate to settle. The rate between 45 seconds and 105 seconds was used as the default measuring period, though occasionally a different period was used os necessary (eg if an aberrant reading was obtained at one of these time-points).

Statistical tests were employed in-house using Graph Pad Prism for this preliminary study (i.e. not using an independent qualified statistician). ANOVA followed by Dunnett's test was used to compare the various time-points with baseline. P<0.05 was considered to be statistically significant. Baseline values were combined for each day (ie n=8) to increase the power of the analysis.

After oral administration, the (R)-3-hydroxybutyrate ester derivatives of the monosaccharides, ie tri- and tetra-(R)-3-hydroxybutyrate ester derivatives of fructose (KTX 0311) and the tri-(R)-3-hydroxybutyrate ester derivative of sorbitol (KTX 0313), were found to produce significant increases in plasma 3-hydroxybutyrate concentrations. In contrast, the tri- and tetra-(R)-3-hydroxybutyrate ester derivatives of glucose (KTX 0310) and the di- and tri-(R)-3-hydroxybutyrate ester derivatives of arabinose (KTX 0312) did not evoke significant ketogenesis in rats after oral administration at the doses and times tested. KTX 0301 (a mixture of a (R)-3-hydroxybutyrate ester derivatives of pullulan and a poly-3-hydroxybutyrate oligomer) also produced significant increases in plasma 3-hydroxybutyrate concentrations after oral administration, whereas KTX 0321 (a different (R)-3-hydroxybutyrate ester derivative of pullulan) and KTX 0322 (a (R)-3-hydroxybutyrate ester derivative of soluble starch) did not evoke significant ketogenesis in rats after oral administration at the doses and times used.

1. A compound of general formula
(R(OCH(CH₃)CH₂C(O))ₙO)ₘ-A
wherein n is an integer between 1 and 10, m is an integer of 1 to 200,000, A is a residue of fructose and R is H,
wherein m is such that the number of free hydroxyl groups on the compound is at least an average of 0.3 free hydroxyls per saccharide moiety in residue A.

2. A compound as claimed in claim 1 wherein m is an integer between 0.5 and 4.

3-6. (canceled)

7. A nutraceutrial composition comprising a compound as claimed in claim 1 together with a foodstuff component.

8. A composition as claimed in claim 7 wherein the foodstuff component is selected from the group consisting of edible oils, emulsions, gels, solids and drinkable liquids.

9. A composition as claimed in claim 8 wherein the foodstuff is selected from the group consisting of drinkable suspensions and solutions.

10. A pharmaceutical composition comprising a compound as claimed in claim 1 together with a pharmaceutically acceptable carrier, diluent or excipient.

11-12. (canceled)

13. A method of treating a patient in need of therapy for one or more of neurodegeneration, diabetes, epilepsy, stroke, head trauma, myocardial infraction, congestive heart failure and obesity comprising administering to that patient a therapeutically effective amount of a compound of claim 1.

14. A method for the manufacture of a compound of formula
R(OCH(CH₃)CH₂C(O))ₙO)ₘ-A
wherein n is an integer between 1 and 10, m is an integer of 1 to 200,000, A is a residue of fructose and R is H,
wherein m is such that the number of free hydroxyl groups on the compound is at least an average of 0.3 free hydroxyls per saccharide moiety in residue A,
comprising reacting (R)-3-hydroxybutyrate or an oligomer thereof containing between 2 and 10 (R)-3-hydroxybutyrate moieties with fructose in the presence of an acid component in an organic solvent.

15. A method as claimed in claim 14 wherein the solvent provides the acid component.

16. A method as claimed in claim 14 wherein the solvent is an organic acid.

17. A method as claimed in claim 14 wherein the solvent is toluene sulphonic acid.

18. A method for the manufacture of a compound of formula
R(OCH(CH₃)CH₂C(O))ₙO)ₘ-A
wherein n is an integer between 1 and 10, m is an integer of 1 to 200,000, A is a residue of fructose and R is H,
wherein m is such that the number of free hydroxyl groups on the compound is at least an average of 0.3 free hydroxyls per saccharide moiety in residue A,
comprising reacting (R)-3-hydroxybutyrate or an oligomer thereof containing between 2 and 10 (R)-3-hydroxybutyrate moieties with fructose in the presence of dimethyl sulphoxide.

19. A method as claimed in claim 14 wherein n in formula 1 is more than 1
comprising reacting fructose having been substituted with H(OCH(CH₃)CH₂C(O))ₙO—, wherein n is 1, with a cyclic oligomer of (R)-hydroxybutyrate in the presence of a lipase in an organic solvent.

20. A method as claimed in claim 19 wherein the solvent is THF and the lipase is CAL B Novozym 435.

21. A method as claimed in claim 19 wherein the cyclic oligomer is (R)-hydroxybutyrate triolide.

22. (canceled)

23. A method as claimed in claim 17 wherein the solvent is para-toluene sulphonic acid.
